(19) United States
(12) Patent Application Publication
(10) Pub. No.: US 2003/0216614 A1
(43) Pub. Date: Nov. 20, 2003

(54) Tip portion of flexible inserting tube of electronic endoscope

(75) Inventor: Takashi Sawai, Tokyo (JP)

Correspondence Address: GREENBLUM & BERNSTEIN, P.L.C., 1950 ROLAND CLARKE PLACE, RESTON, VA, 20191, US

(73) Assignee: PENTAX Corporation, Tokyo (JP)

(21) Appl. No.: 10/438,051

(22) Filed: May 15, 2003

(30) Foreign Application Priority Data
May 16, 2002 (JP) 2002-141015

(51) Int. Cl. A61B 1/04

(52) U.S. Cl. 600/110; 600/129

(57) ABSTRACT

A tip portion of a flexible inserting tube of an electronic endoscope includes a solid-state image sensing device mounted in the tip portion of the flexible inserting tube. The solid-state image sensing device includes a buffer substrate having a rear surface. The rear surface is provided with an earth terminal. The rear surface is also provided with an electronic component protruding rearwards from the rear surface. An conductive block such as a metal block is fixed on the earth terminal. The conductive block protrudes rearwards beyond the electronic component. A signal cable is passed through the flexible tube. The signal cable has a ground line such as a woven metal shield surrounding at least one signal line of the signal cable. The ground line of the signal cable is connected to the conductive block at a side opposite to the buffer substrate.

### BACKGROUND OF THE INVENTION

The present invention relates to a tip portion of a flexible inserting tube of an electronic endoscope that is provided with a solid-state image sensing device at the tip portion.

An Electronic endoscope is provided with a solid-state image sensing device, such as a CCD, at a distal end of a flexible inserting tube thereof in order to capture images of the inside of a body cavity.

FIG. 1 schematically shows the solid-state image sensing device 102 and a distal end of a signal cable 104 (which is to be connected to the solid-state image sensing device 102) of a conventional electronic endoscope. The solid-state image sensing device 102 is provided with a buffer substrate 106 at the rear side thereof. An earth terminal (ground pad) 110 is formed to the rear surface 107 of the buffer substrate 106, which earth terminal 110 is to be connected with a ground line or a woven metal shield 112 of the signal cable 104.

The rear surface 107 of the buffer substrate 106 is also provided with one or more electronic components 108 (only one is shown in FIG. 1). These electronic components 108 inhibit the woven metal shield 112 approaching the rear surface 107 of the buffer substrate 106. Thus, when the woven metal shield 112 is to be connected to the earth terminal 110, the signal cable 104 should be suitably displaced in lateral direction so that the woven metal shield 112 does not interfere with the electronic components 108 on the buffer substrate 106. As a result, the central axis 114 of the signal cable 104 displaces from the center of the solid-state image sensing device 102, 116, for a distance e as shown in FIG. 1. Such displacement of the signal cable 104 makes it difficult to mount the solid-state image sensing device 102 into the flexible inserting tube, which has only a limited space for the solid-state image sensing device and the signal cable.

Therefore, there is a need for an electronic endoscope that is provided with a solid-state image sensing device that allows a signal cable to be connected thereto without requiring laterally shifting the signal cable relative to the solid-state image sensing device.

### SUMMARY OF THE INVENTION

The present invention is advantageous in that a tip portion of a flexible inserting tube of an electronic endoscope and a solid-state image sensing device that satisfy the above mentioned need are provided.

According to an aspect of the invention, a tip portion of a flexible inserting tube of an electronic endoscope is provided that includes a solid-state image sensing device mounted in the tip portion of the flexible inserting tube. The solid-state image sensing device includes a buffer substrate having a rear surface. The rear surface is provided with an earth terminal. The rear surface is also provided with an electronic component protruding rearwards from the rear surface. A conductive block, such as a metal block, is fixed on the earth terminal. The conductive block protrudes rearwards beyond the electronic component. A signal cable is passed through the flexible tube. The signal cable has a ground line such as a woven metal shield surrounding at least one signal line of the signal cable. The ground line of the signal cable is connected to the conductive block at a side opposite to the buffer substrate. Since the conductive block protrudes beyond the electronic component, the ground line can be connected to the conductive block, and in turn to the earth terminal of the buffer substrate, without interfering with the electronic component provided on the buffer substrate irrespective of the lateral position of the signal cable.

Optionally, the earth terminal is formed to the rear surface of the buffer substrate at a location displaced from a center of the buffer substrate, and the ground line of the signal cable is connected to the conductive block with the signal cable being disposed such that a central axis thereof aligns with a center of the buffer substrate.

According to another aspect of the invention, a solid-state image sensing device having front and rear surfaces is provided. The front surface includes an area adapted to receive optical image to be captured. The rear surface is provided with an electronic component. The rear surface is also provided with a ground portion protruding rearwards beyond said electronic component.

Optionally, the ground portion is displaced from a center of the rear surface.

Optionally, the ground portion includes a ground pad formed to the rear surface and a conductive member, which may be a metal block, for example, mounted on the ground pad. The conductive member has a height larger than any of the electronic components in a direction perpendicular to the rear surface.

### BRIEF DESCRIPTION OF THE ACCOMPANYING DRAWINGS

FIG. 1 schematically shows a solid-state image sensing device and a distal end of a signal cable of a conventional electronic endoscope;

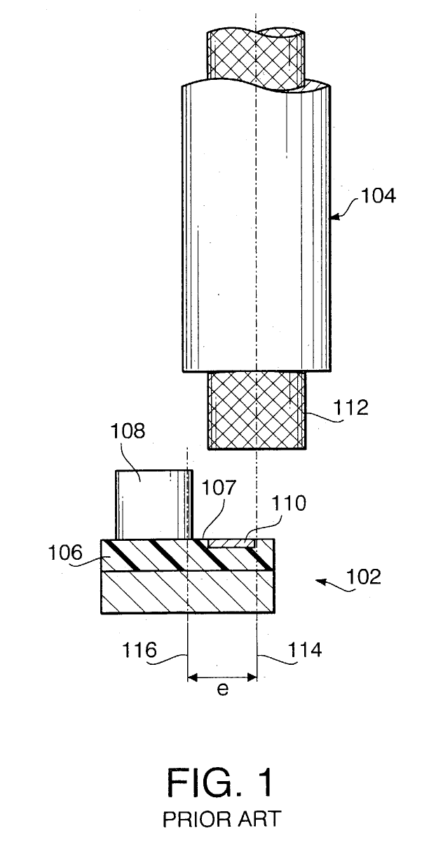

FIG. 2 schematically shows a sectional view of a distal end of a flexible inserting tube of an electronic endoscope according to an embodiment of the invention; and

FIG. 3 shows an enlarged view of a solid state image sensing device of the flexible inserting tube shown in FIG. 2.

### DETAILED DESCRIPTION OF THE EMBODIMENTS

Hereinafter, an embodiment of the present invention will be described with reference to the accompanying drawings.

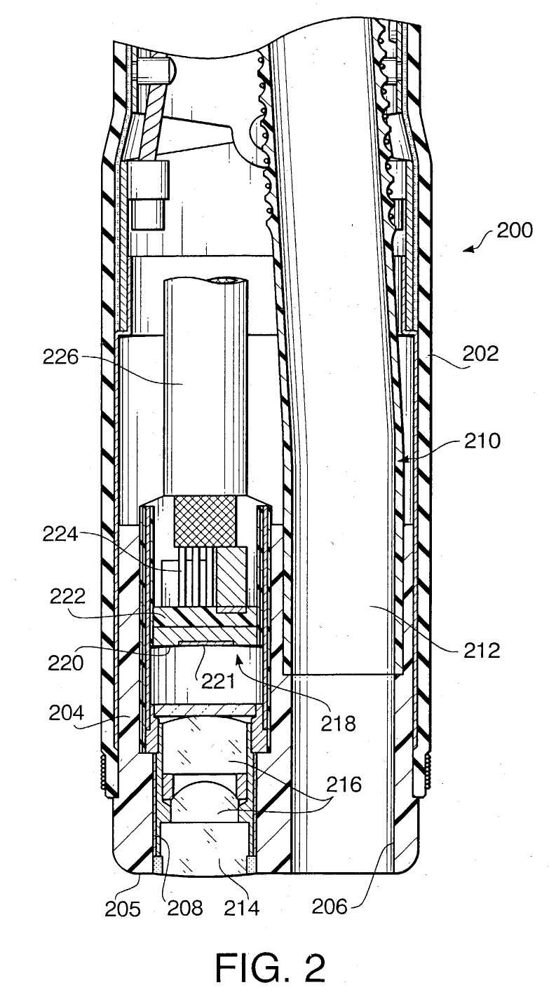

FIG. 2 schematically shows a sectional view of a distal end of a flexible inserting tube 200 of an electronic endoscope according to an embodiment of the invention.

The flexible inserting tube 200 includes an elongated flexible sheath 202 and a rigid tip body 204 attached to a distal end of the sheath 202. The tip body 204 is provided with first and second through holes 206 and 208.

An optical fiber bundle 210 is passed through the flexible inserting tube 200 to provide light for illuminating the inside of a body cavity into which the flexible inserting tube 200 is inserted. A distal end 212 of the optical fiber bundle 210 fits into the first through hole 206 to fix the optical fiber bundle 210 to the tip body 204.

The second through hole 208 of the tip body 204 is provided with an observation window 214, an optical system 216, and a solid-state image sensing device 218, arranged in this turn from the distal end 205 of the tip body 204.

The solid-state image sensing device 218 is located within the second through hole 208 such that an image sensing area 221 formed on a front surface 220 thereof is placed at an image forming plane of the optical system 216.

The solid-state image sensing device 218 is provided with a buffer substrate 222 at a rear side thereof. The buffer substrate 222 is connected with a plurality of signal lines 224 of a signal cable 226 passed through the flexible inserting tube 202.

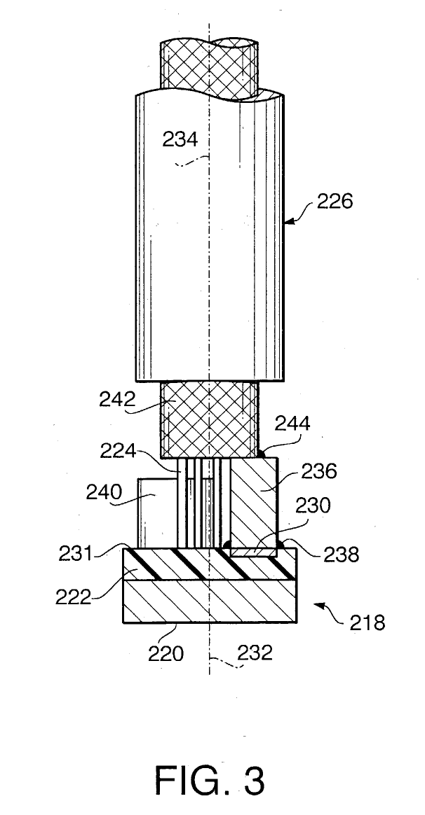

FIG. 3 schematically shows an enlarged view of the solid state image sensing device 218 and the signal cable 226 shown in FIG. 2.

An earth terminal (ground pad) 230 is formed to the rear surface 231 of the buffer substrate 222 at a location displaced from a center line 232 of the solid-state image sensing device 218, which center line 232 perpendicularly penetrates the buffer substrate 222 (or the solid-state image sensing device 218) at a center of the buffer substrate 222 (or of the solid-state image sensing device 218).

A conductive block, such as a metal block 236, is fixed on the earth terminal 230 by means of jointing material 238 such as conductive adhesive or solder. Note that an exemplary material of the metal block 236 is copper alloy.

One or more electronic components 240 are mounted on the rear surface 231 of the buffer substrate 222. The metal block 236 has a height larger than any of the electronic components 240 mounted on the buffer substrate 222. Thus, the metal block 236 protrudes rearwards beyond the electronic components 240 on the buffer substrate 222.

The signal cable 226 has a woven metal shield 242, or a ground line, surrounding the plurality of signal lines 224. The shield 242 is electrically connected with the metal block 236 and hence with the earth terminal 230. More specifically, the shield 242 is abutted against and fixed to the rear end of the metal block 236 by means of jointing material 244 such as conductive adhesive or solder.

In the arrangement above, the shield 242 does not interfere with any of the electronic components 240 on the buffer substrate 222 since the metal block 236 has a height larger than any of the electronic components 240. Thus, the signal cable 226 can be connected to the solid-state image sensing device 218 with the central axis 234 thereof aligned with the center line 232 of the solid-state image sensing device 218, and thereby facilitate mounting of the solid-state image sensing device 218 into the flexible inserting tube 200.

The present disclosure relates to the subject matter contained in Japanese Patent Application No. P2002-141015, filed on May 16, 2002, which is expressly incorporated herein by reference in its entirety.

What is claimed is:

1. A tip portion of a flexible inserting tube of an electronic endoscope, comprising:
a solid-state state image sensing device mounted in said tip portion of said flexible inserting tube, said solid-state image sensing device including a buffer substrate having a rear surface, said rear surface being provided with an earth terminal and an electronic component, said electronic component protruding rearwards from said rear surface;
a conductive block fixed on said earth terminal, said conductive block protruding rearwards beyond said electronic component; and
a signal cable passed through said flexible tube, a ground line of said signal cable being connected to said conductive block at a side opposite to said buffer substrate.

2. The tip portion of the flexible inserting tube of the electronic endoscope according to claim 1,
wherein said earth terminal is formed to said rear surface of said buffer substrate at a location displaced from a center of said buffer substrate, and
wherein said ground line of said signal cable is connected to said conductive block with said signal cable being disposed such that a central axis thereof aligns with a center of said buffer substrate.

3. The tip portion of the flexible inserting tube of the electronic endoscope according to claim 1, wherein said conductive block is a metal block.

4. The tip portion of the flexible inserting tube of the electronic endoscope according to claim 1, wherein said ground line of said signal cable is a woven metal shield surrounding at least one signal line of said signal cable.

5. A solid-state image sensing device, comprising:
a front surface including an area adapted to receive optical image to be captured; and
a rear surface provided with a ground portion and an electronic component, said ground portion protruding rearwards beyond said electronic component.

6. The solid-state image sensing device according to claim 5, wherein said ground portion is displaced from a center of said rear surface.

7. The solid-state image sensing device according to claim 5, wherein said ground portion includes a ground pad formed to said rear surface and a conductive member mounted on said ground pad, said conductive member having a height larger than said electronic component in a direction perpendicular to said rear surface.

8. The solid-state image sensing device according to claim 7, wherein said conductive member is a metal block.
